(19) United States
(12) Patent Application Publication
(10) Pub. No.: US 2015/0359481 A1
(43) Pub. Date: Dec. 17, 2015

(54) METHOD, SYSTEM AND PROGRAM PRODUCT FOR MONITORING OF SLEEP BEHAVIOR

(71) Applicants: Jarrett L. Nyschick, Dearborn Heights, MI (US); Seth Kontny, Davisburg, MI (US)

(72) Inventors: Jarrett L. Nyschick, Dearborn Heights, MI (US); Seth Kontny, Davisburg, MI (US)

(21) Appl. No.: 14/302,039

(22) Filed: Jun. 11, 2014

(51) Int. Cl. A61B 5/00; A61B 5/11; A61B 5/18; A61B 5/0205; G08B 21/06; A61B 5/117

(52) U.S. Cl. CPC A61B 5/4812; G08B 21/06; A61B 5/0077; A61B 5/117; A61B 5/0022; A61B 5/746; A61B 5/18; A61B 5/742; A61B 5/0205; A61B 5/1128; A61B 5/0013; A61B 5/4815; A61B 5/747; A61B 2503/12; A61B 2505/07; A61B 5/02416

(57) ABSTRACT

A method, system and program product comprises instructing a computing device to receive a user inputted information at least identifying the user. The computing device is configured for home usage. A computer vision sensor in communication with the computing device is initialized. The computer vision sensor is configured for home usage. The initializing instructs the computer vision sensor to detect aspects of sleep deprivations of the user. Detected aspects of sleep deprivations of the user during a sleep cycle of the user are received. The computing device is instructed to transmit at least the detected aspects of sleep deprivations of the user to a computer system of a recipient.

### CROSS-REFERENCE TO RELATED APPLICATIONS

The present Utility patent application claims priority benefit of the [U.S. provisional application for patent Ser. No. 61/833,783, filed on 2013 Jun. 11 under 35 U.S.C. 119(e). The contents of this related provisional application are incorporated herein by reference for all purposes to the extent that such subject matter is not inconsistent herewith or limiting hereof.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to monitoring. More particularly, the invention relates to monitoring of sleep behavior.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Some currently available means for monitoring behavior of humans and/or objects may be confusing and difficult for users. Some of these options may require restrictive on-patient equipment, extensive unique monitoring and/or processing equipment, and may be difficult to employ in home, transportation vehicle, and other settings.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. One such aspect of the prior art shows a method for detecting sleep-related apneas, hypopneas, heart rate, body movements, and snoring events of a sleeping person. By way of educational background, another aspect of the prior art generally useful to be aware of teaches systems and methods for providing contact-less sleep disorder diagnosis, including a sound input device and/or a movement detector that receives sound and/or movement data originating from a patient in a sleeping environment. Another such aspect of the prior art discloses a system in which data from load cells may be collected in a person's home, allowing physicians and researchers the ability to monitor a patient's sleep over time without imposing on the patient in their sleep. However, these solutions may not be suitable for providing effective monitoring of behavior using in part common household computer systems. These solutions may also not be suitable for use in transportation vehicle settings. A solution which provided these capabilities would be desirable.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an illustration of an exemplary system for monitoring behavior of people and/or things, in accordance with an embodiment of the present invention;

FIG. 2 is an illustration of an exemplary method for implementing monitoring services from home, in accordance with an embodiment of the present invention;

FIG. 3 is an illustration of an exemplary method for initiating monitoring services from an office, in accordance with an embodiment of the present invention;

FIG. 4 is an illustration of exemplary equipment which may be suitable for collecting sensory data, in accordance with an embodiment of the present invention;

FIG. 5 is an illustration of monitoring zones of embodiment monitoring equipment, in accordance with an embodiment of the present invention;

FIG. 6 is a block diagram depicting an exemplary client/server system which may be used by an exemplary web-enabled/networked embodiment of the present invention; and

FIG. 7 illustrates a block diagram depicting a conventional client/server communication system.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, FireFox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

Some embodiments may be suitable for monitoring behavior of people and/or things. In a non-limiting example, a system may be suitable for use in sleep patient monitoring. Some embodiments may utilize imaging equipment which may read sensory data without requiring on-patient equipment.

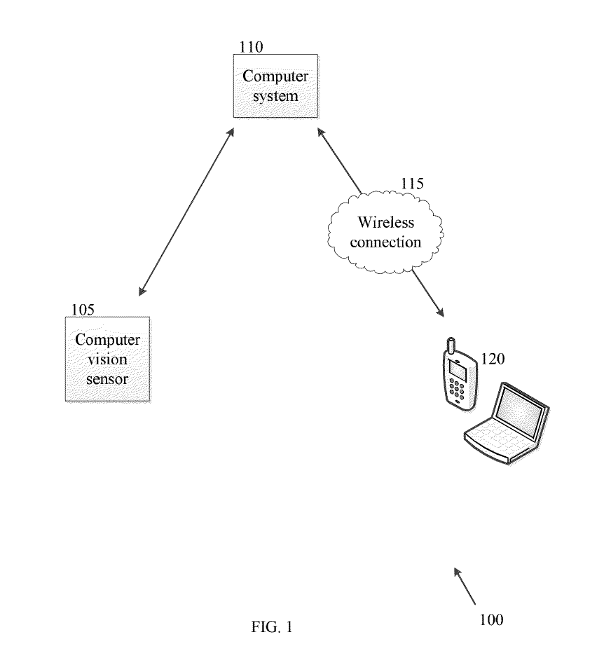

FIG. 1 is an illustration of an exemplary system for monitoring behavior of people and/or things, in accordance with an embodiment of the present invention. In the present embodiment, system 100 may utilize a computer vision sensor 105. In some embodiments, computer vision sensor 105 may be suitable for thermal imaging RGB, in-depth sensing, and video RGB sensing. In a non-limiting example, sensor 105 may be a PrimeSense 3D sensor, Microsoft Kinect sensor, or other suitable device. In the present embodiment, sensor 105 may be linked via a wired and/or wireless connection to a computer system 110. In a non-limiting example, a computer system 110 may be a home entertainment system, such as, without limitation, Xbox, Play Station, etc., or any other suitable system. In some alternative embodiments, smartphones and/or other portable devices may be suitable for use as computer system 110. In some of these alternative embodiments, portable computer system 110 may provide improved ability of performing monitoring in transportation vehicles and/or other areas where more powerful computer equipment may be unavailable. In some embodiments, computer system 110 may have automation functionality for sending and/or receiving data. In the present embodiment, computer system 110 may communicate bi-directionally via a wired and/or wireless connection 115 with various electronic devices 120. In some embodiments, communication may take place via an internet connection 115. In a non-limiting example, sensor 105 may collect sleep monitoring data from a patient in a home. In the present non-limiting example, computer system 110 may receive sleep monitoring data from sensor 105 and may send the data to patient's doctor. Further, in the present non-limiting example, doctor's device 120 may receive sleep monitoring data and may record the data in real-time for evaluation by doctor. In some embodiments, computer system 110 may receive notifications, alerts, messages, and/or other data from devices 120. In a non-limiting example, a doctor may use a device 120 to send alerts, either automatically or manually, to a patient if patient stops breathing or shows any other potentially harmful behavior. In a non-limiting example, computer system 110 may be instructed to produce a loud audible alert. In the present non-limiting example, doctor may also send alerts to emergency medical services for critical events, such as a patient not breathing.

Other currently available systems may require use of SD cards for recording data regarding patient behavior. Some embodiments of the present invention may not require any SD cards, and may instead use real-time data transfer.

Some embodiments may be suitable for use in vehicles. In a non-limiting example, a system may provide sleep monitoring of fleet vehicle operators for sleep amount and efficiency. In the present non-limiting example, data from sleep monitoring may be used by fleet service schedulers to determine optimum hours of safe driving which a fleet driver may perform per day. Further, in the present embodiment, monitored users may first provide a release of medical records prior to monitoring.

Other currently available systems may rely on on-patient equipment which may be detrimental to a patient's sleep improvement outlook. Some embodiments of the present invention may instead use off-patient remote sensors 105 to monitor patients.

In some embodiments, users may download embodiment software onto any suitable electronic device, including, without limitation, smartphones, laptops, tablets, etc. In one embodiment, users may register with software in order to access various features of software. In a non-limiting example, a user may register by providing user's profile information, including user's healthcare provider, in order to establish a recipient for user's sensory data. In some embodiments, software may provide recommendations to users based on user input. In a non-limiting example, if a user states that user suffers from sleep deprivation, software may provide user with options of home monitoring, smart provider directory, medical hardware, etc. In a non-limiting example, if the user does not have a current sleep healthcare provider, but is trying to get more information and find a provider, the system may recommend sleep care providers in the directory within a specified radius of the user's home or work address. Additionally if the user is already a sleep patient with a specific set of equipment that may not be providing the user the optimal benefits, the software may consider current hardware and unresolved sleep issues and through directory search look for other users who have experienced similar issues and received improvements through hardware changes or improvements.

In many embodiments, while patient monitoring may not require on-patient equipment, system may provide on-patient equipment for helping users sleep. In some of these embodiments, system may utilize 3D facial dimensioning and custom mask 3D printing to yield a device that may be custom fit for comfort and prevention of air loss.

Some embodiments of the present invention may provide a variety of advantages over currently available solutions. Some currently available solutions may rely on mass-produced sleep masks and may also rely on on-patient equipment for monitoring purposes. In some instances, on-patient monitoring equipment may be detrimental to a patient's sleep improvement outlook. Some embodiments of the present invention may be suitable for implementing household products such as, without limitation, Kinect and Xbox, for receiving and/or transmitting monitoring data. Many currently available solutions may not be suitable for using such household items.

Some currently available solutions may not provide an integrated virtual location for connecting users. Many embodiments of the present invention may provide a virtual location which may be suitable for connecting a variety of users, including, without limitation, patients, clinics, and doctors, by allowing users to send and/or receive various types of data, including, without limitation, monitoring data, text messages, etc.

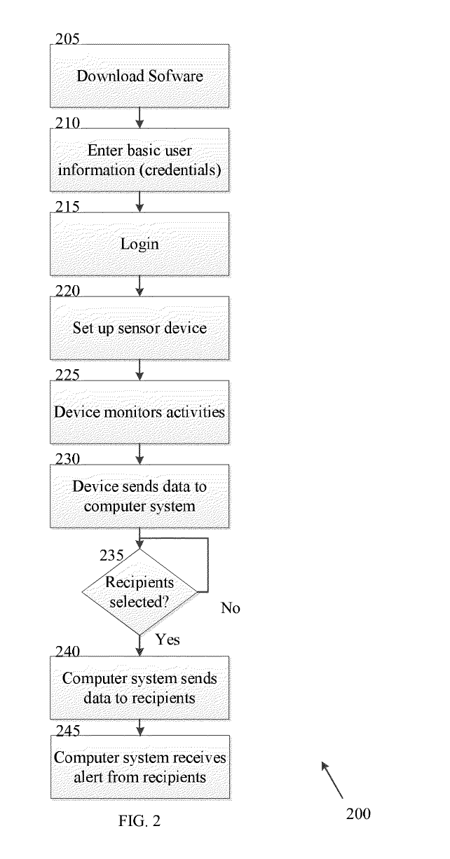

FIG. 2 is an illustration of an exemplary method for implementing monitoring services from home, in accordance with an embodiment of the present invention. Some embodiments may be suitable for use by users of any age and/or gender and for use in any location. In some embodiments, software may be linked to various external software implementation, including, without limitation, Microsoft Vault™. In the present embodiment, users may download application in a step 205. In some embodiments, application may be downloaded onto any device capable of interfacing with a computer vision sensor device 105, including, without limitation, Xbox™, PlayStation™, desktop computer, etc. In the present embodiment, users may enter credentials into software in a step 210. In a non-limiting example, after downloading the application, registration of basic user data such as, but not limited to, name, e-mail address, etc. may be the initial step to explore the software and options. In the present embodiment, users may login to software services in a step 215. In some embodiments, users may register with software prior to being able to login. In a non-limiting example, creating a login (including a password) and logging into the application creates an actual user account where additional user information such as but not limited to, credentials may be entered and stored. In the present embodiment, users may set up computer vision sensor device 105 in a step 220. In some embodiments, software and/or software application may provide an explanation for process of setting up computer vision sensor device 105 to software application and may provide a walkthrough of how to monitor user's activities. In the present embodiment, sensor device 105 may monitor user activities in a step 225. In some embodiments, sensor device 105 may monitor aspects which may pertain to sleep deprivations, including, without limitation, breathing, movement of body, pressure, heart rate, etc. In the present embodiment, sensor device 105 may send monitoring data to computer system 110 in a step 230. In some embodiments, data may be sent in real-time. In many embodiments, received data may be stored in computer system 110. In some embodiments, users may have a personal dashboard in software which may be available to users upon login on any device. In some of these embodiments, dashboard may provide information to users to improve education and awareness. In the present embodiment, software may prompt user to select one or more recipients for user's monitoring data in a step 235. In some embodiments, software may provide users a list of potential recipients. In a non-limiting example, software may use user's location data to provide a list of healthcare providers in user's given area which may have sleep care solutions assistants who may be familiar with embodiment software and/or system. In some embodiments, potential recipients may be selected based on a variety of guidelines. In a non-limiting example, healthcare providers may be required to meet a level of respectfulness towards clients, have a sleep care solutions assistant kiosk within office location, and/or have provider's office registered with software. In some embodiments, potential recipients may sign an agreement before being able to access user information, including, without limitation, user monitoring recordings. In some of these embodiments, potential recipients may only access user data after user has selected recipient. In the present embodiment, after user has chosen a recipient, computer system 110 may send monitoring data to recipient in a step 240. In some embodiments, data may be sent in real-time. In one embodiment, computer system 110 may also send notifications for any unusual events. In a non-limiting example, computer system 110 may send a notification providing any information regarding extended time periods when a user stopped breathing. In some embodiments, monitoring data and/or notifications may appear on recipient's dashboard within software.

In some embodiments, computer vision sensor device 105 may be suitable for creating a virtual representation of persons and/or objects. In a non-limiting example, device 105 may create a virtual representation of a user's face. In some of these embodiments, the virtual representation may be suitable for creating a virtual fitting of an object which may match up with a person and/or object. In a non-limiting example, software may create a virtual prototype of a sleep mask which may be designed to fit a user's face, based on virtual data gathered by a device 105. In the present non-limiting example, the user may need to stand in front of the sensor until the application told the user that facial mapping was complete. The facial mapping may be the only time the user may need to sit/stand in front of the sensor. Sleep monitoring is done as the user sleeps naturally. In the present non-limiting example, a healthcare provider may receive the virtual data and create the sleep mask using a 3D printer. In alternate embodiments, vision sensor device 105 may be used for scanning for dentistry or orthodontics.

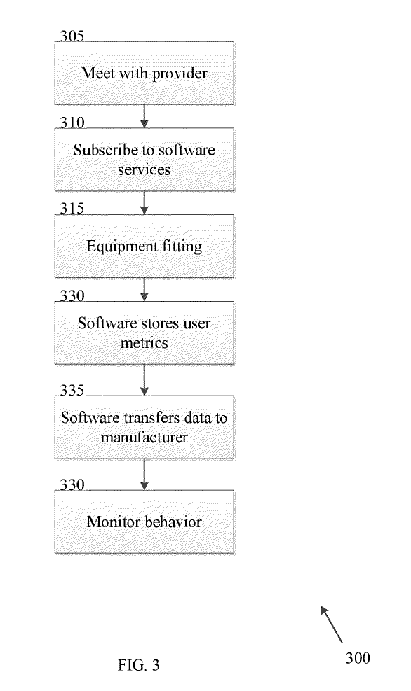

FIG. 3 is an illustration of an exemplary method for initiating monitoring services from an office, in accordance with an embodiment of the present invention. In the present embodiment, a potential user may meet with a provider at an office in a step 305. In a non-limiting example, provider may be a sleep care solutions assistant operating out of a doctor's office. In the present non-limiting example, sleep care solutions assistant may have subscribed to software to become a valid provider and/or recipient of monitoring services. In the present embodiment, user may subscribe to software services in a step 310. In some embodiments, user may provide proof of need for services. In a non-limiting example, user may provide proof that user meets HIPPA compliance and may authorize release of medical records. In the present embodiment, user may be fitted for a sleep mask in a step 315. In some embodiments, software application may provide a means for initiating a facial scan for 3D imaging to create a mapping mesh. In the present embodiment, software stores user metrics in a step 320. In a non-limiting example, user metrics may be stored using a medical records provider such as, without limitation, Microsoft health vault, which may provide necessary data for creation of 3D printed proof of concept mask. In some embodiments, healthcare provider may create a proof-of-concept mask using user metrics in order to ensure that metrics are correct. In the present embodiment, user data may be transmitted to a manufacturing location for manufacturing of custom 3D printed mask in final mask material in a step 325. Further, in the present embodiment, user may begin monitoring behavior using suitable equipment in a step 330. In some embodiments, monitoring may be performed at any location, including, without limitation, user's home or a doctor's office.

In some embodiments, users may access embodiment software from any suitable device, including, without limitation, laptop computer, mobile device, etc. In many embodiments, software may allow access to various resources, including, without limitation, user education resources, user dashboard, provider/recipient dashboard, user metrics (electronic medical record providers), subscription processing software for users and/or providers, etc. In some embodiments, software may provide bi-directional communication between users and providers/recipients, in which users and/or providers may send a variety of types of information, including, without limitation, videos (e.g. video of user while being monitored), sensory data, text messages, images, etc. In a non-limiting example, a doctor may receive sleep monitoring data from a patient and may perform an evaluation on the data. In the present non-limiting example, if doctor determines that patient has a sleep disorder, doctor may use software to send patient an invitation to make an appointment. Further, in the present embodiment, patient may set up an appointment using software. In some embodiments, user may be a friend or relative of a person to be monitored. In some of these embodiments, user may be required to receive authorization from person to be monitored. In one embodiment, system may use Bayesian/based brand intelligence marketing, which may be based on aggregated user data from users who may have had success with sleep monitoring and/or monitoring equipment. Some of these embodiments may provide useful information to users who may be new to the system or may not be having success using the system.

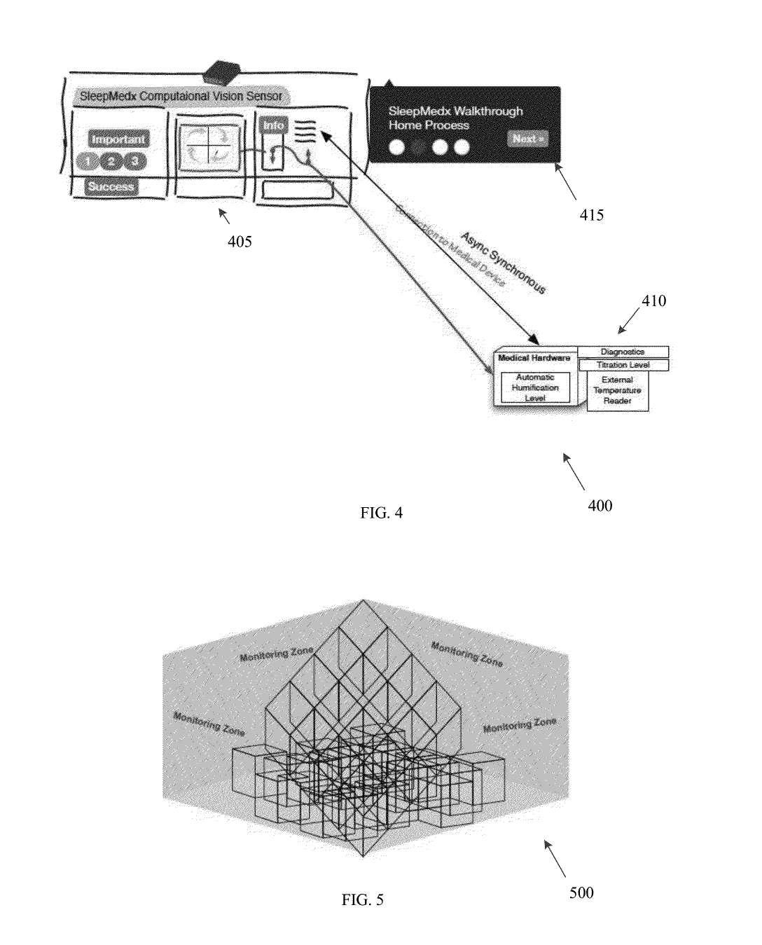

FIG. 4 is an illustration of exemplary equipment which may be suitable for collecting sensory data, in accordance with an embodiment of the present invention. In the present embodiment, a computational vision sensor 405 may communicate bi-directionally with various external equipment 410 to collect sensory data. External equipment 410 may include, without limitation, typical medical hardware that may be required by the user, such as, but not limited to, for proper breathing. In the present embodiment, computational vision sensor 405 may be include, without limitation, PrimeSense 3D sensor, Microsoft Kinect sensor, or other suitable device. In the present embodiment, computational vision sensor 405 may include, without limitation, a computer system which may be a home entertainment system, such as, without limitation, Xbox, Play Station, etc., or any other suitable system. In some alternative embodiments, smartphones and/or other portable devices may be suitable for use as computer system. In some of these alternative embodiments, a portable computer system may provide improved ability of performing monitoring in transportation vehicles and/or other areas where more powerful computer equipment may be unavailable. In a non-limiting example, shown in computational vision sensor 405, are typical displays that may be generated for display such as, but not limited to, important messages, success messages upon registration, etc. Display 415 illustrates a typical display during a walkthrough process. The walkthrough process may include, without limitation, showing the user how to setup the computational vision sensor 405 to begin monitoring with the application. FIG. 5 is an illustration of monitoring zones of embodiment monitoring equipment, in accordance with an embodiment of the present invention. In the present embodiment, monitoring may be accomplished from just one sensor device, but if additional sensors are added to the system, additional coverage area may be achieved. FIG. 5 is a general representation of additional coverage area, but is not meant to represent a specific measured coverage area.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

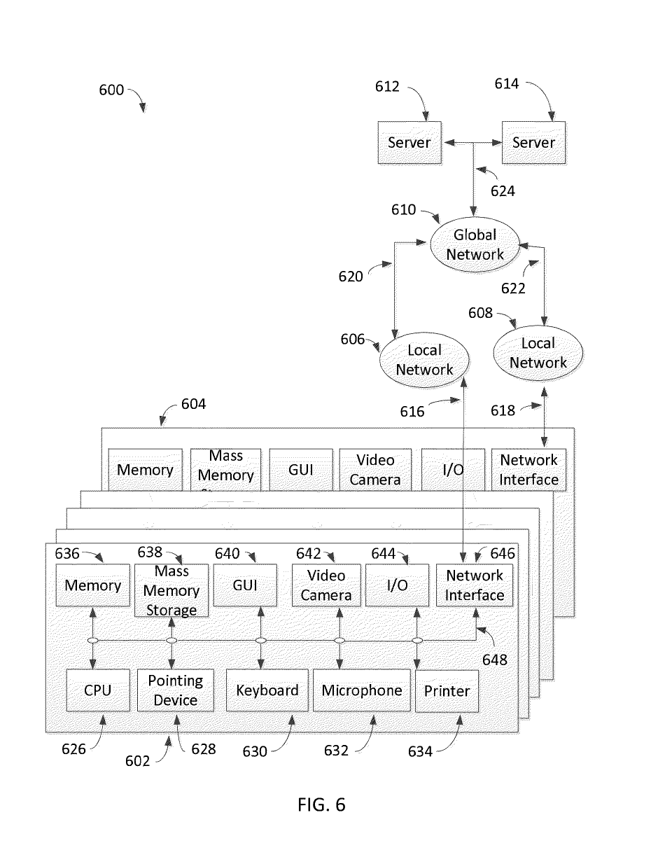

FIG. 6 is a block diagram depicting an exemplary client/server system which may be used by an exemplary web-enabled/networked embodiment of the present invention.

A communication system 600 includes a multiplicity of clients with a sampling of clients denoted as a client 602 and a client 604, a multiplicity of local networks with a sampling of networks denoted as a local network 606 and a local network 608, a global network 610 and a multiplicity of servers with a sampling of servers denoted as a server 612 and a server 614.

Client 602 may communicate bi-directionally with local network 606 via a communication channel 616. Client 604 may communicate bi-directionally with local network 608 via a communication channel 618. Local network 606 may communicate bi-directionally with global network 610 via a communication channel 620. Local network 608 may communicate bi-directionally with global network 610 via a communication channel 622. Global network 610 may communicate bi-directionally with server 612 and server 614 via a communication channel 624. Server 612 and server 614 may communicate bi-directionally with each other via communication channel 624. Furthermore, clients 602, 604, local networks 606, 608, global network 610 and servers 612, 614 may each communicate bi-directionally with each other.

In one embodiment, global network 610 may operate as the Internet. It will be understood by those skilled in the art that communication system 600 may take many different forms. Non-limiting examples of forms for communication system 600 include local area networks (LANs), wide area networks (WANs), wired telephone networks, wireless networks, or any other network supporting data communication between respective entities.

Clients 602 and 604 may take many different forms. Non-limiting examples of clients 602 and 604 include personal computers, personal digital assistants (PDAs), cellular phones and smartphones.

Client 602 includes a CPU 626, a pointing device 628, a keyboard 630, a microphone 632, a printer 634, a memory 636, a mass memory storage 638, a GUI 640, a video camera 642, an input/output interface 644 and a network interface 646.

CPU 626, pointing device 628, keyboard 630, microphone 632, printer 634, memory 636, mass memory storage 638, GUI 640, video camera 642, input/output interface 644 and network interface 646 may communicate in a unidirectional manner or a bi-directional manner with each other via a communication channel 648. Communication channel 648 may be configured as a single communication channel or a multiplicity of communication channels.

CPU 626 may be comprised of a single processor or multiple processors. CPU 626 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, memory 636 is used typically to transfer data and instructions to CPU 626 in a bi-directional manner. Memory 636, as discussed previously, may include any suitable computer-readable media, intended for data storage, such as those described above excluding any wired or wireless transmissions unless specifically noted. Mass memory storage 638 may also be coupled bi-directionally to CPU 626 and provides additional data storage capacity and may include any of the computer-readable media described above. Mass memory storage 638 may be used to store programs, data and the like and is typically a secondary storage medium such as a hard disk. It will be appreciated that the information retained within mass memory storage 638, may, in appropriate cases, be incorporated in standard fashion as part of memory 636 as virtual memory.

CPU 626 may be coupled to GUI 640. GUI 640 enables a user to view the operation of computer operating system and software. CPU 626 may be coupled to pointing device 628. Non-limiting examples of pointing device 628 include computer mouse, trackball and touchpad. Pointing device 628 enables a user with the capability to maneuver a computer cursor about the viewing area of GUI 640 and select areas or features in the viewing area of GUI 640. CPU 626 may be coupled to keyboard 630. Keyboard 630 enables a user with the capability to input alphanumeric textual information to CPU 626. CPU 626 may be coupled to microphone 632. Microphone 632 enables audio produced by a user to be recorded, processed and communicated by CPU 626. CPU 626 may be connected to printer 634. Printer 634 enables a user with the capability to print information to a sheet of paper. CPU 626 may be connected to video camera 642. Video camera 642 enables video produced or captured by user to be recorded, processed and communicated by CPU 626.

CPU 626 may also be coupled to input/output interface 644 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 626 optionally may be coupled to network interface 646 which enables communication with an external device such as a database or a computer or telecommunications or internet network using an external connection shown generally as communication channel 616, which may be implemented as a hardwired or wireless communications link using suitable conventional technologies. With such a connection, CPU 626 might receive information from the network, or might output information to a network in the course of performing the method steps described in the teachings of the present invention.

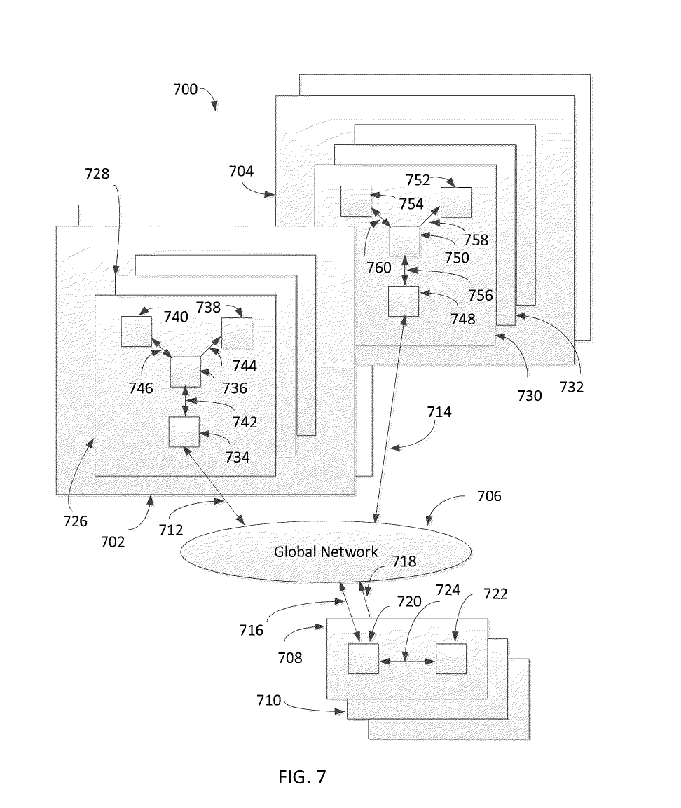

FIG. 7 illustrates a block diagram depicting a conventional client/server communication system.

A communication system 700 includes a multiplicity of networked regions with a sampling of regions denoted as a network region 702 and a network region 704, a global network 706 and a multiplicity of servers with a sampling of servers denoted as a server device 708 and a server device 710.

Network region 702 and network region 704 may operate to represent a network contained within a geographical area or region. Non-limiting examples of representations for the geographical areas for the networked regions may include postal zip codes, telephone area codes, states, counties, cities and countries. Elements within network region 702 and 704 may operate to communicate with external elements within other networked regions or within elements contained within the same network region.

In some implementations, global network 706 may operate as the Internet. It will be understood by those skilled in the art that communication system 700 may take many different forms. Non-limiting examples of forms for communication system 700 include local area networks (LANs), wide area networks (WANs), wired telephone networks, cellular telephone networks or any other network supporting data communication between respective entities via hardwired or wireless communication networks. Global network 706 may operate to transfer information between the various networked elements.

Server device 708 and server device 710 may operate to execute software instructions, store information, support database operations and communicate with other networked elements. Non-limiting examples of software and scripting languages which may be executed on server device 708 and server device 710 include C, C++, C# and Java.

Network region 702 may operate to communicate bi-directionally with global network 706 via a communication channel 712. Network region 704 may operate to communicate bi-directionally with global network 706 via a communication channel 714. Server device 708 may operate to communicate bi-directionally with global network 706 via a communication channel 716. Server device 710 may operate to communicate bi-directionally with global network 706 via a communication channel 718. Network region 702 and 704, global network 706 and server devices 708 and 710 may operate to communicate with each other and with every other networked device located within communication system 700.

Server device 708 includes a networking device 720 and a server 722. Networking device 720 may operate to communicate bi-directionally with global network 706 via communication channel 716 and with server 722 via a communication channel 724. Server 722 may operate to execute software instructions and store information.

Network region 702 includes a multiplicity of clients with a sampling denoted as a client 726 and a client 728. Client 726 includes a networking device 734, a processor 736, a GUI 738 and an interface device 740. Non-limiting examples of devices for GUI 738 include monitors, televisions, cellular telephones, smartphones and PDAs (Personal Digital Assistants). Non-limiting examples of interface device 740 include pointing device, mouse, trackball, scanner and printer. Networking device 734 may communicate bi-directionally with global network 706 via communication channel 712 and with processor 736 via a communication channel 742. GUI 738 may receive information from processor 736 via a communication channel 744 for presentation to a user for viewing. Interface device 740 may operate to send control information to processor 736 and to receive information from processor 736 via a communication channel 746. Network region 704 includes a multiplicity of clients with a sampling denoted as a client 730 and a client 732. Client 730 includes a networking device 748, a processor 750, a GUI 752 and an interface device 754. Non-limiting examples of devices for GUI 738 include monitors, televisions, cellular telephones, smartphones and PDAs (Personal Digital Assistants). Non-limiting examples of interface device 740 include pointing devices, mousse, trackballs, scanners and printers. Networking device 748 may communicate bi-directionally with global network 706 via communication channel 714 and with processor 750 via a communication channel 756. GUI 752 may receive information from processor 750 via a communication channel 758 for presentation to a user for viewing. Interface device 754 may operate to send control information to processor 750 and to receive information from processor 750 via a communication channel 760.

For example, consider the case where a user interfacing with client 726 may want to execute a networked application. A user may enter the IP (Internet Protocol) address for the networked application using interface device 740. The IP address information may be communicated to processor 736 via communication channel 746. Processor 736 may then communicate the IP address information to networking device 734 via communication channel 742. Networking device 734 may then communicate the IP address information to global network 706 via communication channel 712. Global network 706 may then communicate the IP address information to networking device 720 of server device 708 via communication channel 716. Networking device 720 may then communicate the IP address information to server 722 via communication channel 724. Server 722 may receive the IP address information and after processing the IP address information may communicate return information to networking device 720 via communication channel 724. Networking device 720 may communicate the return information to global network 706 via communication channel 716. Global network 706 may communicate the return information to networking device 734 via communication channel 712. Networking device 734 may communicate the return information to processor 736 via communication channel 742. Processor 746 may communicate the return information to GUI 738 via communication channel 744. User may then view the return information on GUI 738.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA. For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC §112 (6) it is intended that the corresponding means for and/or steps for carrying out the claimed function are the ones that are locally implemented within the jurisdiction of the USA, while the remaining aspect(s) performed or located remotely outside the USA are not intended to be construed under 35 USC §112 (6). In some embodiments, the methods and/or system components which may be located and/or performed remotely include, without limitation, remote monitoring of the user.

It is noted that according to USA law, all claims must be set forth as a coherent, cooperating set of limitations that work in functional combination to achieve a useful result as a whole. Accordingly, for any claim having functional limitations interpreted under 35 USC §112 (6) where the embodiment in question is implemented as a client-server system with a remote server located outside of the USA, each such recited function is intended to mean the function of combining, in a logical manner, the information of that claim limitation with at least one other limitation of the claim. For example, in client-server systems where certain information claimed under 35 USC §112 (6) is/(are) dependent on one or more remote servers located outside the USA, it is intended that each such recited function under 35 USC §112 (6) is to be interpreted as the function of the local system receiving the remotely generated information required by a locally implemented claim limitation, wherein the structures and or steps which enable, and breathe life into the expression of such functions claimed under 35 USC §112 (6) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present application, and “35 USC §112 (6)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing monitoring according to the present invention will be apparent to those skilled in the art. Various aspects of the invention have been described above by way of illustration, and the specific embodiments disclosed are not intended to limit the invention to the particular forms disclosed. The particular implementation of the monitoring may vary depending upon the particular context or application. By way of example, and not limitation, the monitoring described in the foregoing were principally directed to sleep monitoring implementations; however, similar techniques may instead be applied to monitoring of any human and/or object behavior, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A method comprising the steps of:
instructing a computing device to receive a user inputted information at least identifying the user, the computing device being configured for home usage;
initializing a computer vision sensor being in communication with the computing device, the computer vision sensor being configured for home usage, said initializing instructing the computer vision sensor to detect aspects of sleep deprivations of the user;
receiving detected aspects of sleep deprivations of the user during a sleep cycle of the user; and
instructing the computing device to transmit at least the detected aspects of sleep deprivations of the user to a computer system of a recipient.

2. The method as recited in claim 1, further comprising the step of instructing the computing device issue an alert to the user upon receipt of an alert from the computer system of the recipient.

3. The method as recited in claim 1, further comprising the step of instructing the computer vision sensor to perform a three-dimensional image scanning of the user for facilitating equipment fitting.

4. The method as recited in claim 3, instructing the computing device to transfer metrics of the three-dimensional scanning to a location for fabrication.

5. The method as recited in claim 1, in which the recipient is a fleet service scheduler using the transmitted aspects for determining hours of safe driving for the user.

6. The method as recited in claim 1, in which the aspects are transmitted in real time.

7. The method as recited in claim 1, in which said step of initializing a computer vision sensor further comprises instructing the computing device to display a walkthrough of a setup to the user.

8. The method as recited in claim 2, in which the alert from the computer system of the recipient is further sent to emergency medical services.

9. The method as recited in claim 1, in which the detection of the aspects of sleep deprivations occurs at the user's home.

10. The method as recited in claim 1, in which the detection of the aspects of sleep deprivations is accomplished without physically contacting the user.

11. A system comprising:
a computer vision sensor being configured for home usage, said computer vision sensor being further configured to detect aspects of sleep deprivations of a user at the user's home without physically contacting the user, said aspects comprising at least the user's breathing, movement of body, and heart rate; and
a computing device being configured for home usage, said computing device being further configured to receive inputted information from the user at least identifying the user, instructing the computer vision sensor to detect the aspects of sleep deprivations of the user during a sleep cycle of the user, transmit at least the detected aspects of sleep deprivations of the user to a computer system of a recipient in real time, and issue an alert to the user upon receipt of an alert from the computer system of the recipient.

12. A non-transitory computer-readable storage medium with an executable program stored thereon, wherein the program instructs one or more processors to perform the following steps:
instructing a computing device to receive a user inputted information at least identifying the user, the computing device being configured for home usage;
initializing a computer vision sensor being in communication with the computing device, the computer vision sensor being configured for home usage, said initializing instructing the computer vision sensor to detect aspects of sleep deprivations of the user;
receiving detected aspects of sleep deprivations of the user during a sleep cycle of the user; and
instructing the computing device to transmit at least the detected aspects of sleep deprivations of the user to a computer system of a recipient.

13. The program instructing the processor as recited in claim 12, further comprising the step of instructing the computing device issue an alert to the user upon receipt of an alert from the computer system of the recipient.

14. The program instructing the processor as recited in claim 12, further comprising the step of instructing the computer vision sensor to perform a three-dimensional image scanning of the user for facilitating equipment fitting.

15. The program instructing the processor as recited in claim 14, instructing the computing device to transfer metrics of the three-dimensional scanning to a location for fabrication.

16. The program instructing the processor as recited in claim 12, in which the recipient is a fleet service scheduler using the transmitted aspects for determining hours of safe driving for the user.

17. The program instructing the processor as recited in claim 12, in which the aspects are transmitted in real time.

18. The program instructing the processor as recited in claim 12, in which said step of initializing a computer vision sensor further comprises instructing the computing device to display a walkthrough of a setup to the user.

19. The program instructing the processor as recited in claim 13, in which the alert from the computer system of the recipient is further sent to emergency medical services.

20. The program instructing the processor as recited in claim 12, in which the detection of the aspects of sleep deprivations occurs at the user's home.
